(19) United States
(12) Patent Application Publication
(10) Pub. No.: US 2025/0269148 A1
(43) Pub. Date: Aug. 28, 2025

(54) GUIDE WIRE TIP PROTECTION DEVICE

(71) Applicant: BIOTEQUE CORPORATION, Taipei (TW)

(72) Inventors: MING-CHUNG LEE, Taipei (TW); CHIN-LUNG LIN, Taipei (TW); CHING-YI HSIEH, Taipei (TW); ZHEN-KAI QIAN, Taipei (TW); ZONG-MING LU, Taipei (TW); CHIH-HSIEN CHIEN, Taipei (TW); PEI-LING TSAI, Taipei (TW)

(21) Appl. No.: 18/857,601

(22) PCT Filed: Apr. 25, 2022
(86) PCT No.: PCT/CN2022/089021

§ 371 Date: Oct. 17, 2024

(51) Int. Cl. A61M 25/09; B65D 59/08; B65D 85/04

(52) U.S. Cl. CPC A61M 25/09; B65D 59/08; B65D 85/04; A61M 2209/06

(57) ABSTRACT

The present invention relates to a guide wire tip protection device, comprising a bottom plate, wherein a snap hole, a transverse groove and a through hole are formed on the bottom plate; and an upper cover having a shape corresponding to the shape of the bottom plate, wherein one side edge of the upper cover is connected to the bottom plate, and the upper cover is integrally formed with the bottom plate as a sheet body, the transverse length of the upper cover is greater than that of the bottom plate, and a snap and at least one longitudinal groove are formed on the upper cover.

### CROSS-REFERENCE TO RELATED INVENTIONS

This invention patent application is a national stage entry under 35 U.S.C. 371 and claims the benefit of International Invention Application No. PCT/CN2022/089021, filed on Apr. 25, 2022. The entire contents of which are hereby incorporated herein by reference thereto.

### BACKGROUND OF THE INVENTION cl 1. Field of the Invention

The present invention relates to a protection device, and especially to a protection device configured for protecting a tip of a medical guide wire.

### 2. Description of Related Art

A medical guide wire is a thin, flexible tool used in catheter interventions. Inserted into veins, it guides catheters to central circulation veins, enabling procedures like intravenous infusion, drug administration, and venous pressure measurement. It enhances safety and efficacy in vascular procedures. The guide wire has a tip, so during transporting of the guide wire, a protection device is usually disposed on the tip of the guide wire to prevent the tip of the guide wire from being damaged or deformed due to collision. A conventional protection device, e.g. a guide wire tip protector disclosed by US 20050234369 A1, is a component made of plastic. The protector includes a proximal end and a distal end. The distal end includes a cavity sized to accept a curved distal end of a guide wire and a distal end of a guide wire advancer. The proximal end of the guide wire has an elongated portion and a ring portion. The elongated portion is configured to engage the guide wire advancer. The ring portion is capable of surrounding and combining with tubes. Whereby a function of protecting the tip of the guide wire is achieved.

However, because the aforementioned guide wire tip protector is made of plastic, it requires additional mold designing and mold making, which is costly and time-consuming. The protector made of plastic is not environmentally friendly, and is prone to cause environmental pollution after being discarded. In addition, because an entire structure of the protector made of plastic is solid, it can only be installed on a specified guide wire. In order to mount on a guide wire of a different specification, a new protector needs to be additionally designed and manufactured. In order to fetch out and use the guide wire, a user needs to remove the guide wire protector, and then take the guide wire out, so the tip of the guide wire is prone to damage while being taken out. Therefore, the entire structure of the conventional guide wire tip protector has the foregoing problems and shortcomings to be improved.

### SUMMARY OF THE INVENTION

In view of the shortcomings of the prior arts, the present invention provides a guide wire tip protection device that achieves a purpose of receiving a tip of a guide wire therein by an upper cover being foldable relative to a bottom plate to form a surrounded inner space.

In order to achieve the foregoing invention purpose, the technical means adapted in the present invention is to design a guide wire tip protection device comprising:
a bottom plate having a longitudinal direction and a transverse direction, a snap hole, a transverse groove, and a through hole; the snap hole formed at a side edge of the bottom plate; the transverse groove recessed in a side surface of the bottom plate and extending along the transverse direction; the through hole located at a terminal of the bottom plate and adjacent to the transverse groove;
an upper cover corresponding to the bottom plate in shape; a side edge of the upper cover connected to a side edge, of the bottom plate, away from the snap hole to be integrally formed with the bottom plate as a sheet; a transverse length of the upper cover being larger than a transverse length of the bottom plate; the upper cover having a snap and at least one longitudinal groove; the snap corresponding to the snap hole in position and located at an opposite side edge of the upper cover; the longitudinal groove recessed in a side surface of the upper cover and extending along the longitudinal direction;
wherein, when the snap is inserted into the snap hole and the upper cover is bent along said longitudinal groove, an inner space is formed and surrounded by the upper cover and the bottom plate.

Furthermore, in the guide wire tip protection device mentioned above, the at least one longitudinal groove includes multiple said longitudinal grooves spaced from one another.

Furthermore, in the guide wire tip protection device mentioned above, an upper side edge is defined at one of two opposite ends of the bottom plate; a lower side edge is defined at the other one of the two opposite ends of the bottom plate. A length of the upper side edge is greater than a length of the lower side edge. The through hole is located between the transverse groove and the lower side edge.

Furthermore, in the guide wire tip protection device mentioned above, two cutting lines are transversally cut through the bottom plate apart and parallel to each other. The through hole communicates with each cutting line.

Furthermore, in the guide wire tip protection device mentioned above, the snap is an L-shaped sheet extending outwardly from the opposite side edge of the upper cover and extending along the longitudinal direction. The snap hole is an elongated hole extending along the longitudinal direction. The snap is selectively inserted into or separated from the snap hole.

Furthermore, in the guide wire tip protection device mentioned above, the bottom plate and the upper cover are made of paper materials.

The present invention has the advantage that the upper cover is foldable relative to the bottom plate, so a tip of a guide wire may be inserted into the through hole and be located above the bottom plate, and the upper cover is folded and closed for receiving the tip of the guide wire inside the inner space surrounded by the bottom plate and the upper cover, thereby achieving the function of protecting the tip. To take the tip out, the upper cover can be folded reversely to an opposite side surface, the bottom plate is bent along the transverse groove by an angle, so the tip is spaced apart from the bottom plate to be conveniently taken out and free from being damaged by collision.

### BRIEF DESCRIPTION OF THE DRAWINGS

The following drawings are only intended to illustrate and explain the present invention, and do not limit the scope of the present invention. In addition, the shapes and proportional dimensions of each component in the figures are only schematic and are used to help the understanding of the present invention, and are not intended to specifically limit the shapes and proportional dimensions of each component of the present invention. Under the teaching of the present invention, those skilled in the art can select various possible shapes and proportional sizes according to specific circumstances to implement the present invention.

FIG. 1 is a perspective view of a guide wire tip protection device in accordance with the present invention;

FIG. 2 is an expanded view of the guide wire tip protection device in FIG. 1;

FIG. 3 is an operational schematic diagram of the guide wire tip protection device in FIG. 1;

FIG. 4 is another folded schematic diagram of the guide wire tip protection device in FIG. 1;

FIG. 5 is another operational schematic diagram of the guide wire tip protection device in FIG. 1;

FIG. 6 is another operational side view of the guide wire tip protection device in FIG. 1; and

FIG. 7 is a perspective view of another embodiment of a guide wire tip protection device in accordance with the present invention.

### DETAILED DESCRIPTION OF PREFERRED EMBODIMENTS

The technical means adapted by the present invention to achieve the intended purpose of the invention are illustrated by the drawings and the preferable embodiments of the present invention as follows.

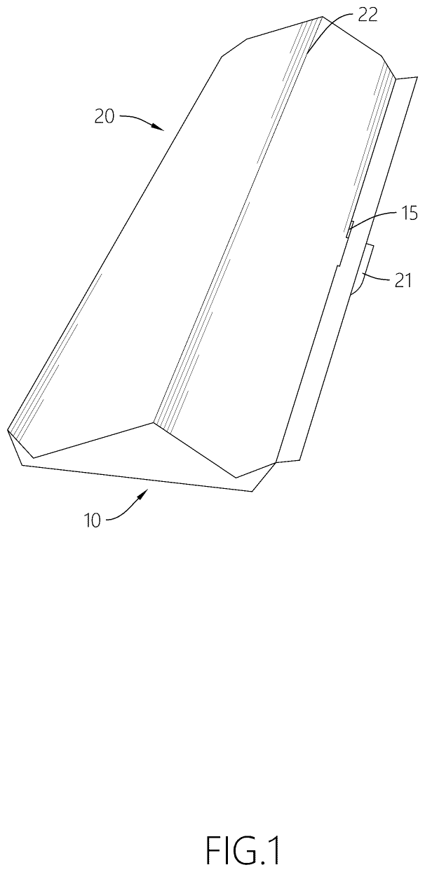

With reference to FIG. 1, a guide wire tip protection device in accordance with the present invention comprises a bottom plate 10 and an upper cover 20.

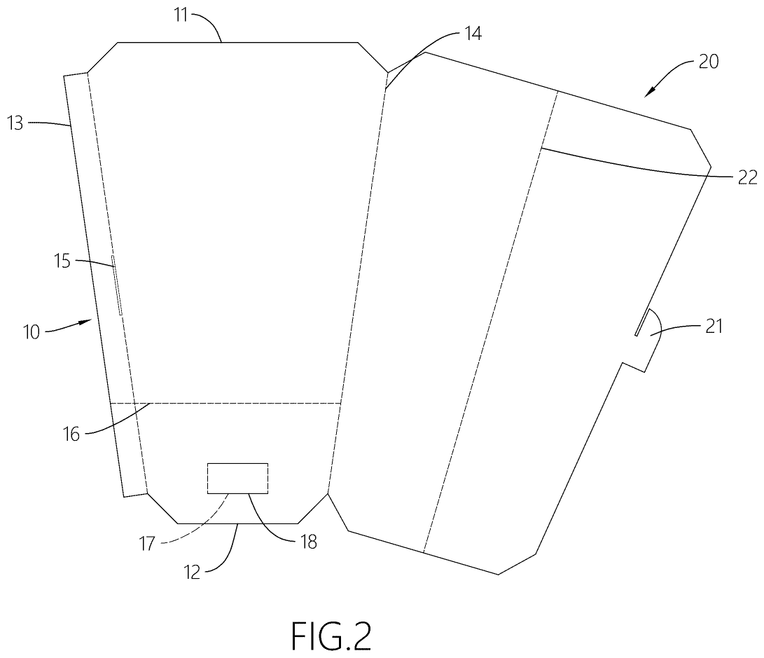

With reference to FIGS. 1 and 2, the bottom plate 10 is a sheet and has a longitudinal direction and a transverse direction. The longitudinal direction and the transverse direction are two directions being perpendicular to each other. The bottom plate 10 has an upper side edge 11 at one of two opposite ends thereof and a lower side edge 12 at the other one of the two opposite ends. An outer side edge 13 and an inner side edge 14 are located between the upper side edge 11 and the lower side edge 12 and respectively at opposite two sides of the bottom plate 10. The bottom plate 10 has a snap hole 15, a transverse groove 16, and a through hole 17 formed therein. The snap hole 15 is formed through the bottom plate 10 at the outer side edge 13 of the bottom plate 10 and is an elongated hole extending along the longitudinal direction. The transverse groove 16 is recessed in a side surface of the bottom plate 10 and extends along the transverse direction. The transverse groove 16 is adjacent to the lower side edge 12. The through hole 17 is a tunnel formed by two cutting lines 18 transversally cut through the bottom plate 10 apart and parallel to each other and communicating with the two cutting lines 18. Specifically, when a segment between the two parallel cutting lines 18 is lifted up, a communicating hole is formed at where the two cutting lines 18 are. The through hole 17 is located between the transverse groove 16 and the lower side edge 12. In the embodiment, the bottom plate 10 is made of paper material. A length of the upper side edge 11 is larger than a length of the lower side edge 12. An overall shape of the bottom plate 10 is similar to a sector, but it is not limited to this. The type of the bottom plate 10 can be changed according to the user's needs. For example, it can be made of other environmentally friendly and recyclable materials, e.g. bioplastics made from wheats or corns.

The upper cover 20 corresponds to the bottom plate 10 in shape and material. A side edge of the upper cover 20 is connected to the inner side edge 14 of the bottom plate 10. The upper cover 20 and the bottom plate 10 are formed as an integral sheet by cutting. A transverse length of the upper cover 20 is larger than a transverse length of the bottom plate 10. In other words, a width of the upper cover 20 is larger than a width of the bottom plate 10. The upper cover 20 has a snap 21 and a longitudinal groove 22. The snap 21 corresponds to the snap hole 15 in position. The snap 21 is an L-shaped sheet outwardly extending from an opposite side edge of the upper cover 20 and extending along the longitudinal direction, and is selectively inserted into or separated from the snap hole 15. The combination of the snap 21 and the snap hole 15 is prior art, and details thereof are omitted. The longitudinal groove 22 is recessed in a side surface of the upper cover 20 and extends along the longitudinal direction. In the embodiment, the longitudinal groove 22 is located at an imaginary central line of the upper cover 20, but it is not limited thereto. The type of the upper cover 20 can be changed according to the user's needs.

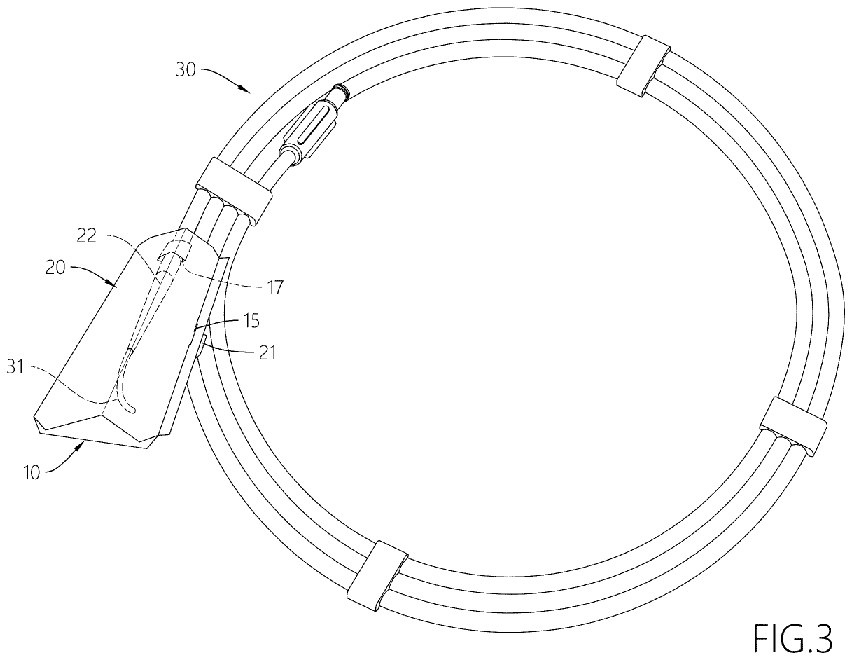

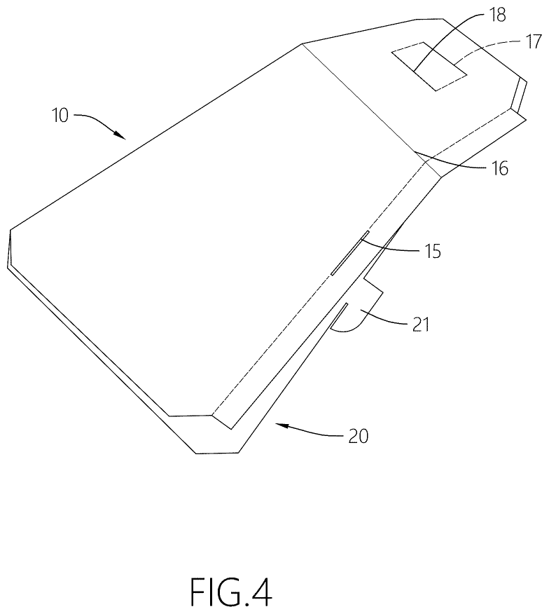

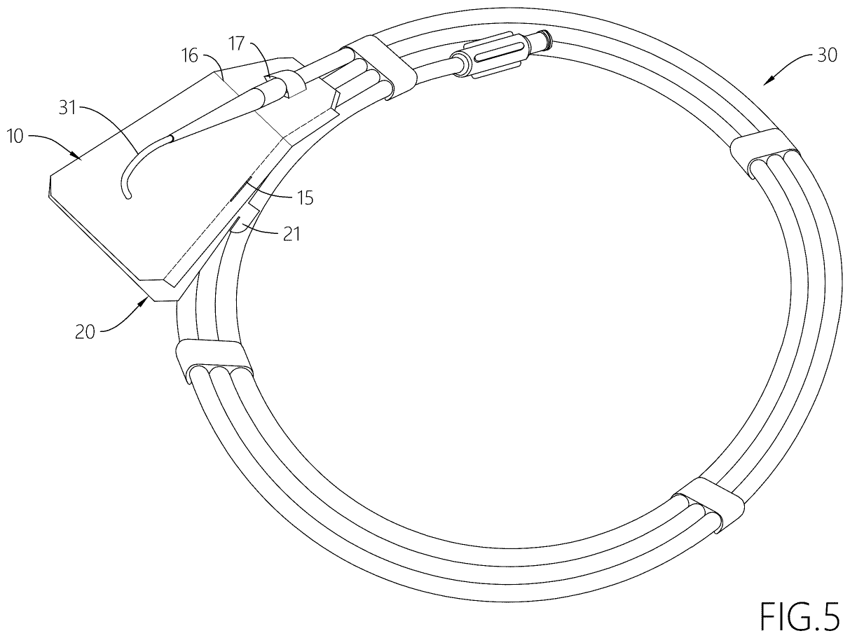

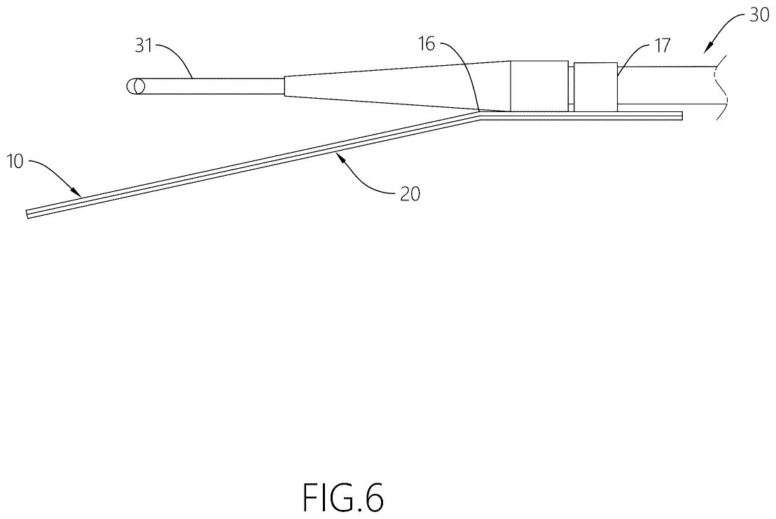

When the present invention is in usage, with reference to FIG. 3 illustrated by applying a guide wire 30 thereto, the guide wire 30 has a tip 31. A user inserts the tip 31 via one of the cutting lines 18 of the bottom plate 10, through the through hole 17, and throughout the other one of the cutting lines 18 to mount the tip 31 through the through hole 17 and at the side surface of the bottom plate 10. The segment formed between the two cutting lines 18 is configured to surround and stably secure the tip 31. The upper cover 20 is bent toward the bottom plate along the longitudinal groove 22 to form an angle, and then the upper cover 20 is folded around the side edge connected to the bottom plate 10 to insert the snap 21 into the snap hole 15 to connect the snap 21 and the snap hole 15 with each other. Whereby the upper cover 20 attaches to the outer side edge 13 of the bottom plate 10 to surround an inner space therein. The tip 31 is disposed in the inner space. Therefore, the effect of protecting the tip 31 is achieved by folding and closing the upper cover 20 and the bottom plate 10 relative to each other to surround the tip 31. With reference to FIGS. 4 to 6, to take the guide wire 30 out, the user separates the snap 21 from the snap hole 15 and fold the upper cover 20 to the other side surface of the bottom plate 10, and then bends the bottom plate 10 along the transverse groove 16 to the side surface opposite to the guide wire 30. Whereby, a part of the bottom plate 10 and a part of the upper cover 20 are spaced apart from the tip 31 of the guide wire 30 to form a space for conveniently reaching the user' hand into the space to take the guide wire 30 out. The tip 31 of the guide wire 30 is prevented from being damaged due to collision while being taken out.

In the aforementioned process, the present invention is made of paper material, which is easily and conveniently accessible. Compared with the prior art made of plastic, designs and manufactures of molds are not required in the present invention, whereby development period is shortened, production cost is reduced, and volume may be reduced during transportation to reduce transportation cost and increase carrying capacity.

In the aforementioned process, since the paper material overcomes the shortcoming of plastic waste being difficult to decompose, the paper material does not produce plastic particles to cause serious damage and pollution in the ecological environment. Therefore, using the paper material is environmentally friendly and may reduce the dependence of petrochemical raw materials.

In the aforementioned process, since the space and the shape surrounded and formed by the bottom plate 10 and the upper cover 20 are suitable for the tips 31 of most of the guide wires 30 on the market, the guide wire 30 accommodated is not restricted by the angle, the dimensions, and numbers of its bends, and is good in versatility.

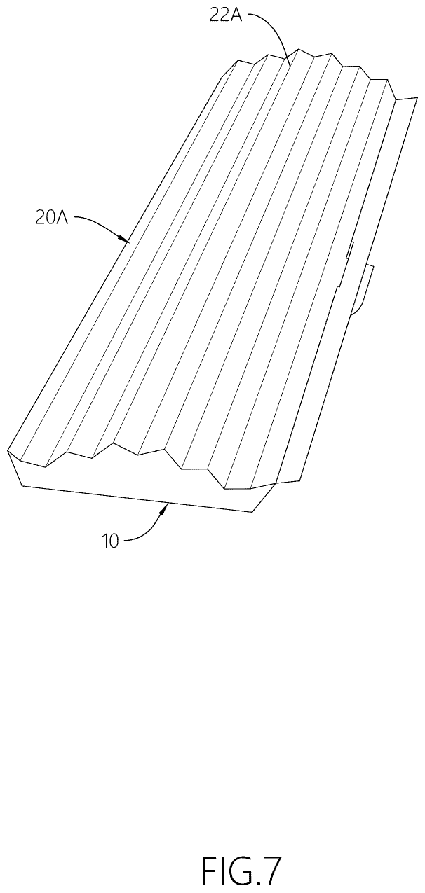

With reference to FIG. 7, in other embodiments, the upper cover 20A may have multiple longitudinal grooves 22A spaced apart from one another. When in use, the upper cover 20A may be folded along each longitudinal groove 22A to form a wavy shape. The structural rigidity of the upper cover 20A is enhanced by the wavy upper cover 20A. The strength of the upper cover 20A to resist external force applied thereto is enhanced.

The above are only the preferred embodiments of the present invention and do not limit the present invention in any form. Although the present invention has been disclosed as above in the preferred embodiments, it is not intended to limit the present invention. Anyone familiar with the professional technology, without departing from the scope of the technical solution of the present invention, can make use of the technical content disclosed above to make slight changes or modification into equivalent embodiments with equivalent changes, but any content that does not depart from the technical solution of the present invention is based on the present invention. Any simple modifications, equivalent changes and modifications made to the above embodiments by technical essence still fall within the scope of the technical solutions of the present invention.

What is claimed is:

1. A guide wire tip protection device, characterized in that the guide wire tip protection device comprises:
a bottom plate having a longitudinal direction, a transverse direction, a snap hole, a transverse groove, and a through hole; the snap hole located at a side edge of the bottom plate; the transverse groove recessed in a side surface of the bottom plate and extending along the transverse direction; the through hole located at a terminal of the bottom plate and adjacent to the transverse groove;
an upper cover corresponding to the bottom plate in shape; a side edge of the upper cover connected to a side edge, of the bottom plate, away from the snap hole to be integrally formed with the bottom plate as a sheet; a transverse length of the upper cover being larger than a transverse length of the bottom plate; the upper cover having a snap and at least one longitudinal groove; the snap corresponding to the snap hole in position and located at an opposite side edge of the upper cover; the longitudinal groove recessed in a side surface of the upper cover and extending along the longitudinal direction;
wherein when the snap is inserted into the snap hole and the upper cover is bent along said longitudinal groove, an inner space is formed and surrounded by the upper cover and the bottom plate.

2. The guide wire tip protection device as claimed in claim 1, characterized in that, the at least one longitudinal groove includes multiple said longitudinal grooves spaced from one another.

3. The guide wire tip protection device as claimed in claim 1, characterized in that, an upper side edge is defined at one of two opposite ends of the bottom plate; a lower side edge is defined at the other one of the two opposite ends of the bottom plate; a length of the upper side edge is greater than a length of the lower side edge; and the through hole is located between the transverse groove and the lower side edge.

4. The guide wire tip protection device as claimed in claim 1, characterized in that, two cutting lines are transversally cut through the bottom plate apart and parallel to each other; and the through hole communicates with each cutting line.

5. The guide wire tip protection device as claimed in claim 1, characterized in that, the snap is an L-shaped sheet extending outwardly from the opposite side edge of the upper cover and extending along the longitudinal direction; the snap hole is an elongated hole extending along the longitudinal direction; the snap is selectively inserted into or separated from the snap hole.

6. The guide wire tip protection device as claimed in claim 1, characterized in that, the bottom plate and the upper cover are made of paper materials.

7. The guide wire tip protection device as claimed in claim 2, characterized in that, an upper side edge is defined at one of two opposite ends of the bottom plate; a lower side edge is defined at the other one of the two opposite ends of the bottom plate; a length of the upper side edge is greater than a length of the lower side edge; and the through hole is located between the transverse groove and the lower side edge.

8. The guide wire tip protection device as claimed in claim 2, characterized in that, two cutting lines are transversally cut through the bottom plate apart and parallel to each other; and the through hole communicates with each cutting line.

9. The guide wire tip protection device as claimed in claim 2, characterized in that, the snap is an L-shaped sheet extending outwardly from the opposite side edge of the upper cover and extending along the longitudinal direction; the snap hole is an elongated hole extending along the longitudinal direction; the snap is selectively inserted into or separated from the snap hole.

10. The guide wire tip protection device as claimed in claim 2, characterized in that, the bottom plate and the upper cover are made of paper materials.
